(19) United States
(12) Patent Application Publication
(10) Pub. No.: US 2007/0016084 A1
(43) Pub. Date: Jan. 18, 2007

(54) Catherter for measuring an intraventricular pressure and method of using same

(76) Inventor: Andre Denault, Longueuil (CA)

Correspondence Address: Louis Tassier, P O Box 54029, Town of Mount-Royal, QC, H3P 3H4, CA

(21) Appl. No.: 10/569,508

(22) PCT Filed: Aug. 30, 2004
(86) PCT No.: PCT/CA04/01584

§ 371 Date: Feb. 27, 2006

Related U.S. Application Data
(60) Provisional application No. 60/498,358, filed on Aug. 28, 2003.
(60) Provisional application No. 60/498,606, filed on Aug. 29, 2003.

(51) Int. Cl. A61B 5/02

(52) U.S. Cl. 600/485; 600/486

(57) ABSTRACT

A method for diagnosing a right ventricular dysfunction of a subject. The method includes measuring a right intraventricular pressure waveform in the subject over at least one cardiac cycle, extracting a ventricular parameter indicative of a right ventricular function from the measured right intraventricular pressure waveform, and establishing a diagnosis at least in part on a basis of the ventricular parameter.

### FIELD OF THE INVENTION

The present invention relates to a catheter for measuring a pressure and a method of using same. Specifically, the present invention concerns a catheter for measuring an intraventricular pressure and a method of using same.

### BACKGROUND OF THE INVENTION

The major cause of death after cardiac surgery is hemodynamic instability. There are specific factors that can predispose a patient to hemodynamic instability. These factors are related to the inability of the heart to relax and accept or receive blood, which is called diastolic dysfunction. When the heart experiences diastolic dysfunction, it requires a higher pressure to be filled, which in some cases leads to serious problem such as pulmonary edema or cardiac malfunction. The latter manifests itself as hemodynamic instability that can lead to death.

There are several types and causes of hemodynamic instability that can occur alone or in combination¹. A few are presented hereinbelow:

Reduced left and right ventricular contractility, caused by:
Myocardial ischemia related complication (intra or extracardiac rupture, reduced function);
Intraoperative coronary occlusion (air, clot, calcium);
Coronary graft malfunction (vascular spasm);
Myocardial depression from extra-cardiac factors (brain injury, sepsis); and
Suboptimal cardioplegia.

Increased left and right ventricular afterload, caused by:
Primary or secondary pulmonary hypertension;
Left ventricular outflow tract obstruction (after mitral repair or aortic surgery; presence of left ventricular hypertrophy);
Acute aortic dissection from the aortic canulation; and
Right outflow ventricular tract obstruction (mechanical in off-pump bypass surgery or dynamic with right ventricular hypertrophy);
Pulmonary embolism (air, clot, carbon dioxide); and
Hypoxia from pulmonary edema or from right-to-left shunt due to patent foramen ovale.

Abnormal left and right ventricular filling:
Myocardial left and right ventricular diastolic dysfunction;
Abnormal left ventricular filling from right ventricular dilatation or pulmonary hypertension; and
Extra-cardiac limitation to cardiac filling (pericardial tamponade, positive-pressure ventilation, thoracic tamponade, abdominal compartment syndrome).

Reduced preload:
Reduced systemic vascular resistance (drugs, sepsis, hemodilution, anaphylaxis); and
Blood losses (external, thoracic, gastro-intestinal, retroperitoneal).

Valvular insufficiency:
Mitral valve insufficiency from ischemia, LVOT obstruction, sub-optimal repair, complication of aortic valve surgery;
Aortic valve insufficiency after mitral valve surgery, dysfunctional prosthesis, aortic dissection; and
Tricuspid valve insufficiency from right ventricular failure.

Echocardiography is the method of choice to diagnose and quantify systolic and diastolic function²⁻⁴. The hypothesis that patients with diastolic dysfunction are at higher risk of hemodynamic instability after cardiac surgery was supported by a pilot study of Bernard et al that included 66 patients of whom 52 had Coronary Artery Bypass Grafting (CABG) alone⁵.

The factors associated with an increased need for vasoactive support after CardioPulmonary Bypass (CPB) were female sex, diastolic dysfunction and prolonged duration of CPB. Diastolic dysfunction was more important than systolic dysfunction in predicting Difficult Separation from Bypass (DSB) and vasoactive requirement after surgery. These findings were reconfirmed by another group of investigators⁶ and supported a by a recent study⁷ of patients with reduced left ventricular systolic function (Left Ventricular Ejection Fraction (LVEF)<=25%) with or without reduced right ventricular dysfunction before coronary revascularization followed up to 4 years.

Patients with reduced LVEF without right ventricular dysfunction and left ventricular diastolic dysfunction had less inotrope requirements after revascularization and a mortality of 9.7%. In patients with reduced LVEF but with reduced right ventricular function (in which 6/7 had a restrictive diastolic function), death occurred in all patients within 18 months (5 patients died during hospitalization).

The associations between pre-operative right ventricular systolic dysfunction and outcomes continued to be statistically significant after pre- and intraoperative covariables were controlled in multivariate regression analysis. This study supports the hypothesis that right ventricular systolic dysfunction is a predictor of mortality before cardiac surgery.

Unfortunately, echocardiography is a highly specialized method that requires extensive knowledge in the interpretation of the data obtained through the technique. In addition, echocardiography requires that a specific procedure be performed on patients that are often already monitored using one or more other techniques. Furthermore, echocardiography is a procedure that is not very suitable for monitoring a patient.

In another context, it is sometimes beneficial for a patient to receive a volume of liquid, such as a saline solution of other to improve cardiac function. However, there are situations, for example in case of a right ventricular diastolic dysfunction, when this injection of volume is not beneficial and is even nocive. Accordingly, having a method for rapidly determining if a patient would benefit from an administration of such a liquid would greatly improve treatment of some patient.

In view of the above, there is a need in the industry to provide novel and improved catheters for measuring an intraventricular pressure and methods of using same.

### SUMMARY OF THE INVENTION

In a first broad aspect, the invention provides a pressure monitoring device for monitoring a right intraventricular pressure in a heart having a right ventricle, the right ventricle having electrically excitable tissues and electrically non-excitable tissues, the right ventricle being in fluid communication with a pulmonary artery. The device includes a pressure measuring portion for measuring the right intraventricular pressure, the pressure measuring portion being insertable within the right ventricle. The device further includes a stabilizer connected to the pressure measuring portion for stabilizing the pressure monitoring device such that when the pressure measuring portion is positioned within the right ventricle for measuring the intraventricular pressure therein. The pressure monitoring device is spaced from the electrically excitable tissues of the right ventricle.

Advantageously, the device allows taking measurements of intraventricular pressure with minimal risks of injuries and other complications, such as arrhythmias, for the subject.

In another broad aspect, the invention provides a method for diagnosing a right ventricular dysfunction of a subject. The method includes the steps of:
measuring a right intraventricular pressure waveform in the subject over at least one cardiac cycle;
extracting a ventricular parameter indicative of a right ventricular function from the measured right intraventricular pressure waveform; and
establishing a diagnosis at least in part on a basis of the ventricular parameter.

The method takes advantage of the common insertion of intracardiac catheters to add a functionality to this type of catheter to measure additional parameters that are of clinical importance. For example, the direct measurement of intraventricular pressure without the need to use echocardiography is simpler and more cost-effective.

In yet another broad aspect, the invention provides a method for monitoring a right ventricular function of a subject having a right ventricle, the method comprising the steps of:
inserting a pressure monitoring device in the right ventricle of the subject;
measuring a right intraventricular pressure waveform in the subject over a plurality of cardiac cycles; and
extracting a ventricular parameter indicative of a right ventricular function from the measured waveform for at least some cardiac cycles from the plurality of cardiac cycles.

In yet another broad aspect, the invention provides a method for classifying a subject as being likely to experience complications during a surgery, the method including the steps of
measuring a right intraventricular pressure waveform in the subject over at least one cardiac cycle prior to the surgery;
extracting a ventricular parameter indicative of a right ventricular function from the measured waveform; and
establishing a likelihood of occurrence of complications during the surgery at least in part on a basis of the ventricular parameter.

Other objects, advantages and features of the present invention will become more apparent upon reading of the following non restrictive description of preferred embodiments thereof, given by way of example only with reference to the accompanying drawings.

### BRIEF DESCRIPTION OF THE DRAWINGS

In the appended drawings:

FIG. 1 is illustrates schematically a pressure monitoring device inserted in the heart of a subject;

FIG. 2A is a schematic cross-section of a pressure monitoring device;

FIG. 2B is a top elevation view of the pressure monitoring device of FIG. 2A;

FIG. 3 is a is a schematic cross-section of an alternative pressure monitoring device;

FIG. 4 illustrates a model of the pathophysiology of hemodynamic instability in cardiac surgical patients;

FIG. 5 illustrates right intraventricular waveforms in patients that were respectively responsive and non-responsive to the administration of 500 mL of a colloidal solution;

FIG. 6 illustrates a right ventricular outflow tract obstruction in a 75 years-old man after coronary revascularization and aortic valve replacement. A trans-gastric mid-papillary short-axis echographic view revealed a dilated and hypertrophied right ventricle. Unexplained acute right heart failure was present without pulmonary hypertension. Pulmonary artery, arterial and right intraventricular pressure waveforms are also shown.

FIG. 7 illustrates a an echocardiogram and a continuous Doppler ultrasound signal for the same patient as in FIG. 6;

FIG. 8 illustrates a mid-esophageal right ventricular inflow-outflow view exam and a M-mode echocardiography for the same patient as in FIG. 6;

FIG. 9 compares an hemodynamic and a transesophageal echocardiographic evaluation of a 46 yrs old woman scheduled for aortic valve endocarditis; and

FIG. 10 compares a right intaventricular pressure waveform and an hepatic Doppler signal in a 81 years old female scheduled for coronary revascularization, aortic and mitral valve replacement.

### DESCRIPTION OF THE PREFERRED EMBODIMENTS

Introductory Remarks

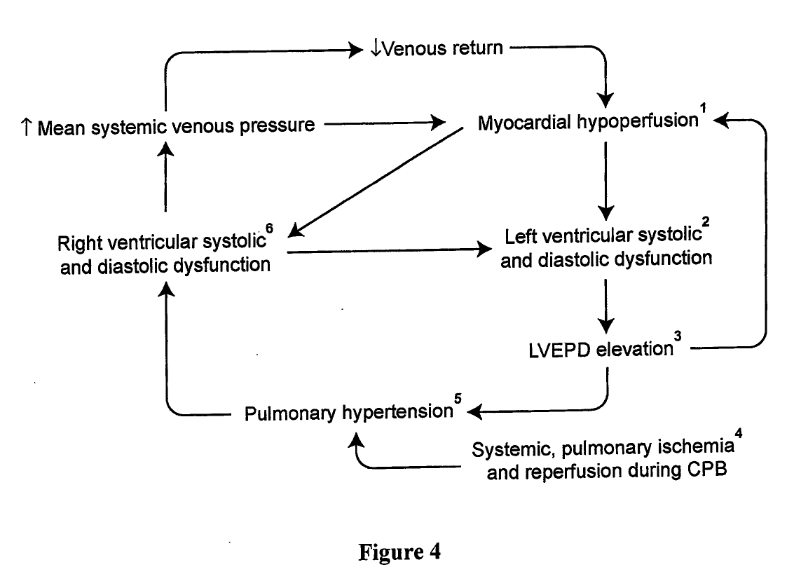

From available animal and human clinical data, the following pathophysiological model of hemodynamic instability in cardiac surgical patients, illustrated in FIG. 4, is produced.

Myocardial hypoperfusion leads and predisposes to systolic and diastolic dysfunction. With progression of the phenomenon, elevation in Left Ventricular End Diastolic Pressure (LVEDP) occurs, which in turn may lead to secondary pulmonary hypertension and right ventricular systolic and diastolic dysfunction. Pulmonary hypertension is also be exacerbated with the pulmonary ischemia reperfusion injury after CardioPulmonary Bypass CPB and the inflammatory response to the CPB circuit and the effect of pre-operative or intraoperative tissue hypoperfusion.

In addition, through interventricular interdependence, pulmonary hypertension exacerbates left ventricular diastolic dysfunction leading to more pulmonary hypertension. The final result is a progressive reduction in venous return and cardiac output though increased right sided pressures and signs of right sided failure with associated hemodynamic instability.

Therefore, from the above and from published studies, the following hypotheses on hemodynamic instability after cardiac surgery are formulated:

1—Increased veno-arterial Carbon Dioxyde partial pressure (P_{CO2}) before (CPB) is an independent factor for difficult separation from bypass (DSB)⁸.

2—Left ventricular diastolic dysfunction⁹ and right ventricular diastolic dysfunction predisposes to hemodynamic instability and DSB.

3—Elevated LVEDP predisposes to hemodynamic instability, DSB and death¹⁰.

4—Pulmonary ischemia and reperfusion during CPB is associated with pulmonary hypertension and prevented by inhaled prostacyclin¹¹ and global ischemia during CPB increases hemodynamic instability and death⁸.

5—Pulmonary hypertension predisposes to hemodynamic instability¹⁴. Inhaled prostacyclin reduces pulmonary hypertension and the incidence of hemodynamic instability^{12 13}.

6—Right ventricular systolic and diastolic dysfunction is commonly present in hemodynamic instability¹⁵.

Myocardial hypoperfusion chronically or acutely, before and after CPB either through coronary artery disease, poor myocardial protection, clots, air or carbon dioxide embolism during the cardiac procedure and poor cardiac output could lead and predispose to systolic and diastolic dysfunction. As the disease progresses, gradual elevation in LVEDP and secondary pulmonary hypertension¹⁶ may ensue. Pulmonary hypertension may be exacerbated by ischemia reperfusion after CPB and pre-operative or intraoperative global and regional hypoperfusion.

Pulmonary hypertension will eventually lead to progressive right atrial^{17 18} and ventricular dilatation which is associated with abnormal right ventricular systolic and diastolic function. In addition, through ventricular interdependence and ventricular septal shift, pulmonary hypertension could exacerbate left ventricular diastolic dysfunction¹⁹ leading to more severe pulmonary hypertension. The final result is a progressive reduction in venous return and cardiac output through increased right sided pressures and signs of right sided failure with associated hemodynamic instability.

Accurate routine measurement and monitoring of intraventricular pressure has the potential to significantly improve the prognostic for cardiac surgeries and many other interventions. It also presents a perfect opportunity to relatively easily provide diagnostic information.

Pressure Monitoring Device

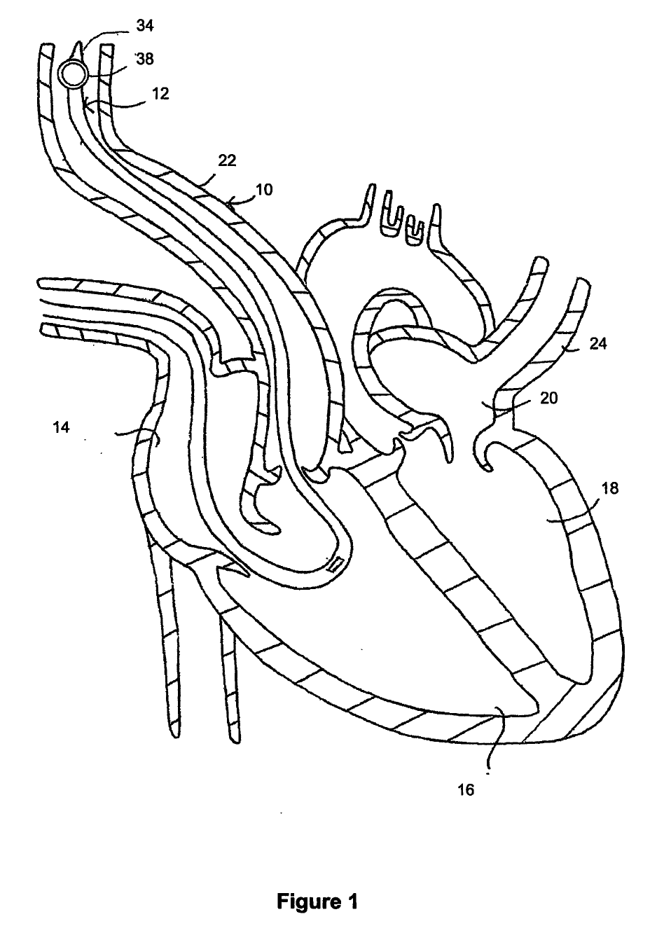

FIG. 1 illustrates schematically the anatomy of the heart 10 of a subject into which a part of a pressure monitoring device 12 is inserted for monitoring a right intraventricular pressure in the heart 10.

The heart 10 includes a right atrium 14, a right ventricle 16, a left atrium 20 and a left ventricle 18. The right atrium 14 is adjacent to and in fluid communication with the right ventricle 16. Similarly, the left atrium 20 is adjacent to and in fluid communication with the left ventricle 18. A pulmonary artery 22 is connected to the right ventricle 16. The left atrium 20 is connected to a pulmonary vein 24. Between the pulmonary artery 22 and the pulmonary vein 24, the lungs (not shown in the drawings) exchange gases between the blood contained within blood vessels and air contained within the lungs. Another part of the blood circulation, namely the systemic circulation, is neither shown in the drawings nor described.

The right atrium 14, the right ventricle 16, the left atrium 20 and the left ventricle 18 each include electrically excitable tissues and electrically non-excitable tissues. The pressure monitoring device 12 includes a pressure measuring portion for measuring the right intraventricular pressure, the pressure measuring portion being insertable within the right ventricle 16. The pressure monitoring device 12 further includes a stabilizer connected to the pressure measuring portion for stabilizing the pressure monitoring device such that when the pressure measuring portion is positioned for measuring the right intraventricular pressure, the pressure monitoring 12 device is spaced from the electrically excitable tissues of the right ventricle.

Accordingly, complications such as arrhythmias and injuries to the right ventricle 18 that could occur if the pressure monitoring device 12 contacted the electrically excitable tissues are minimized.

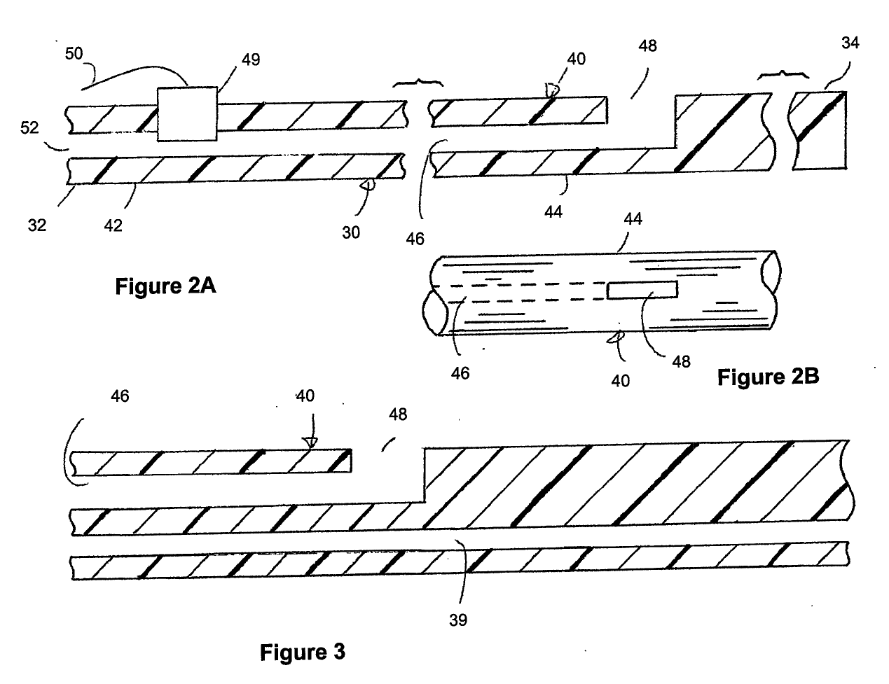

As better seen in FIG. 1 and in FIG. 2A, the stabilizer includes a substantially elongated and deformable stabilizing body 30 defining a proximal stabilizing body end 32 and a longitudinally opposed stabilizing body distal end 34. The stabilising body 30 is located at least in part within the pulmonary artery 22 when the pressure measuring portion is positioned for measuring the intraventricular pressure.

In some embodiments of the invention, as shown in FIG. 1, the stabilizing body distal end 34 is located within the pulmonary artery 22 when the pressure measuring portion is positioned for measuring the intraventricular pressure. In alternative embodiments of the invention, the stabilizing body distal end 34 is not located within the pulmonary artery 22 when the pressure measuring portion is positioned for measuring the intraventricular pressure.

In some embodiments of the invention, the stabilizer includes an inflatable balloon 38 located in proximity to the stabilizing body distal end 34 and an inflation system connected to the balloon 38 for controllably inflating and deflating the balloon.

As shown in FIG. 3, in this case a conduit 39 extends within the pressure monitoring device for conducting a fluid used to inflate and deflate the balloon. The conduit 39 is connected (not shown in the drawings) at one extremity to the balloon 38 and at an opposite extremity to a fluid injection and withdrawal device (not shown in the drawings) that allows to controllably inflate and deflate the balloon.

Such inflatable balloons and associated systems are well-known in the art and will therefore not be described in further details.

In alternative embodiments of the invention, as shown in FIGS. 2A and 2B, the stabilizer does not include an inflatable balloon.

The pressure measuring portion includes a substantially elongated and deformable pressure measurement body 40 defining a pressure measurement body proximal end 42 and a longitudinally opposed pressure measurement body distal end 44, the pressure measurement body distal end 44 being connected to a stabilizing body proximal end 32.

As shown in FIGS. 2A and 2B, but not in FIG. 1, the pressure measurement body has a substantially longitudinally extending lumen 46 and a lateral opening 48 (shown in FIG. 1) in fluid communication with the lumen 46 and extending substantially radially therefrom in proximity to the pressure measurement body distal end 44. In addition, the pressure measurement body 40 includes a pressure sensor 49 for sensing a pressure of a fluid within the lumen 46.

In some embodiments of the invention, as shown in FIG. 2, the pressure sensor 48 is located in proximity to the pressure measurement body proximal end 42 such as to be located outside of the subject when the pressure measuring portion is positioned for measuring the intraventricular pressure. In alternative embodiment of the invention, a pressure sensor is located in proximity to the opening 48.

The pressure sensor 49 is connected to a signal transmission line 50 that transmit an electrical signal indicative of a measured pressure, and produced by the pressure sensor 49, to a suitable interface device (not shown in the drawings).

In some embodiments of the invention, the interface device displays graphically the measured pressure as a function of time. In alternative embodiments of the invention, the interface device prints the measured pressure as a function of time on a suitable medium, such as paper, for example. In other alternative embodiments of the invention, the interface device displays numerical values indicative of the measured pressure. In yet other embodiments of the invention, the interface device displays parameters in the form of numerical values indicative of the measured pressure. For example, the interface device displays a maximal measured pressure for each cardiac cycle.

In some embodiments of the invention, the interface device stores the measured pressures as a function of time on a computer-readable storage medium. In other embodiments of the invention, this functionality is not provided by the interface device.

As shown in FIG. 2B, the opening 48 is substantially rectangular and oriented substantially longitudinally with respect to the pressure measurement body 40. However, openings having any other suitable shape are within the scope of the invention.

In some embodiments of the invention, as shown in FIG. 2, the pressure measurement body 40 includes an injection port 52 located in proximity to the pressure measurement body proximal end 32, the fluid injection port 52 being in fluid communication with the lumen. The fluid injection port 52 is for injecting a fluid within the lumen, the fluid transmitting a pressure at the opening 48 to the pressure sensor 49. The pressure sensor 49 contacts the fluid and therefore measures the pressure transmitted by the fluid. The fluid is any suitable fluid, such as for example, a saline solution. In some embodiments of the invention, the fluid is an isotonic saline solution.

In some embodiments of the invention, the opening 48 is located at about 25-40 cm from the stabilizing body distal end 34, an in some cases at about 30 cm from the stabilizing body distal end 34. However, depending on the geometry of the stabilizing body, the opening 48 is located at any other suitable location in alternative embodiments of the invention.

The pressure measurement body 40 and the pressure sensor 49 are any suitable body and pressure sensors. Specifically, the pressure measurement body 40 is selected such as to have appropriate pressure transmission properties, including a suitable frequency response, and a suitable flexibility allowing threading of the pressure measurement body 22 within the cardiac cavities (atrium and ventricle) of the subject. Also, the pressure sensor 49 is also selected such as to exhibit suitable pressure measurement parameters, for example a suitable frequency response and a suitable sensitivity, among others.

The pressure monitoring device 12 includes any suitable materials, such as for example a biocompatible polymer, among others.

The reader skilled in the art will readily that there are many methods of using the above-described device, depending on the medical condition of the subject and the desired measurements, among others.

Method—Rationale

As mentioned hereinabove, right ventricular systolic dysfunction is a predictor of mortality before cardiac surgery. Thus, following hypothesis is formulated: right ventricular diastolic dysfunction is also a predictor of morbidity and mortality before cardiac surgery.

Pilot studies are supporting such a possibility. As it is well-known, the evaluation of right ventricular diastolic function can be performed by interrogating the hepatic venous flow with pulsed-wave Doppler^{7 8}. In a pilot study of 121 patients undergoing cardiac surgery it was observed<that abnormal hepatic venous flow was associated with separation from bypass requiring more vasoactive support (P<0.05). In a subset of patients undergoing only valvular surgery, abnormal hepatic venous flow before surgery was associated with a higher Parsonnet's score (P=0.0005), more atrial fibrillation (P<0.0001), pacemaker requirement (P=0.0124), mitral valve replacement (P=0.0325), reoperation (P=0.0050), a lower Mean Arterial Pressure/Mean Pulmonary Artery Pressure MAP/MPAP ratio (P=0.0127), a higher wall motion score index (P=0.0491) and a higher incidence of abnormal right ventricular systolic function (P=0.0139).

However abnormal hepatic venous flow before cardiac surgery was not found to be an independent predictor of DSB and worse outcome. In that pilot study, pulmonary hypertension or the MAP/MPAP ratio was the best predictor of hemodynamic complications (Caricard et al in press).

A more recent study from 179 consecutive patients using newer echocardiographic technology suggests that both moderate to severe left and right ventricular diastolic dysfunction are predictors of DSB^{20 21}. These studies include observations on demographic, biochemical, surgical, hemodynamic and echocardiographic variables demonstrate the utility and prognostic nature of these variables. These variables should be viewed as complementary but not exclusive. However, few of the demographic and surgical variables can be modified before cardiac surgery. Only the MAP/MPAP ratio, left and right ventricular systolic and diastolic function represent potential variables that can be altered prior to bypass.

Consequently the diagnosis of right ventricular diastolic dysfunction with a pressure monitoring device in the form of a pulmonary artery catheter, or in any other alternative form, has a potential to identify patients at increased risk of post-operative hemodynamic instability. In addition, it could identify the presence of abnormal right ventricular function which itself could contribute to hemodynamic instability.

As mentioned hereinabove, there are several causes of hemodynamic instability that often occur in combination. Diastolic dysfunction has been found to be the most common echocardiographic abnormality in these hemodynamically unstable patients and importantly, right filling abnormalities were more common than left ventricular diastolic dysfunction.

Right ventricular diastolic dysfunction can be diagnosed using both hemodynamic and echocardiographic criteria. The hemodynamic criteria are obtained through continuous monitoring of the right intraventricular pressure waveform through a pulmonary artery catheter and the echocardiographic criteria from the analysis of trans-tricuspid blood flow, hepatic venous flow and interrogation of the tricuspid annulus using tissue Doppler. In addition, from pilot studies, it appears that the most common denominator in hemodynamic instability is pulmonary hypertension better defined as an reduced MAP/MPAP ratio. Since the study of Costachescu et al and the use of new echocardiographic modalities such as tissue Doppler and color Mmode, it was able to reconfirm that right ventricular diastolic dysfunction is present more commonly in hemodynamically unstable patients after cardiac surgery.

In addition, the use of the continuous right intraventricular pressure waveform monitoring allow the recognition of right ventricular outflow tract obstruction which can happen during cardiac surgery either off-pump bypass or after any type of cardiac surgery. Six patients with such a condition were identified. The presence of such an abnormality potentially contribute to hemodynamic instability.

In view of the above, some examples of use of the above-described pressure monitoring device 12 are described hereinbelow. However, the reader skilled in the art will readily appreciate that these methods do not necessarily require the use of this device and are performed using any suitable device.

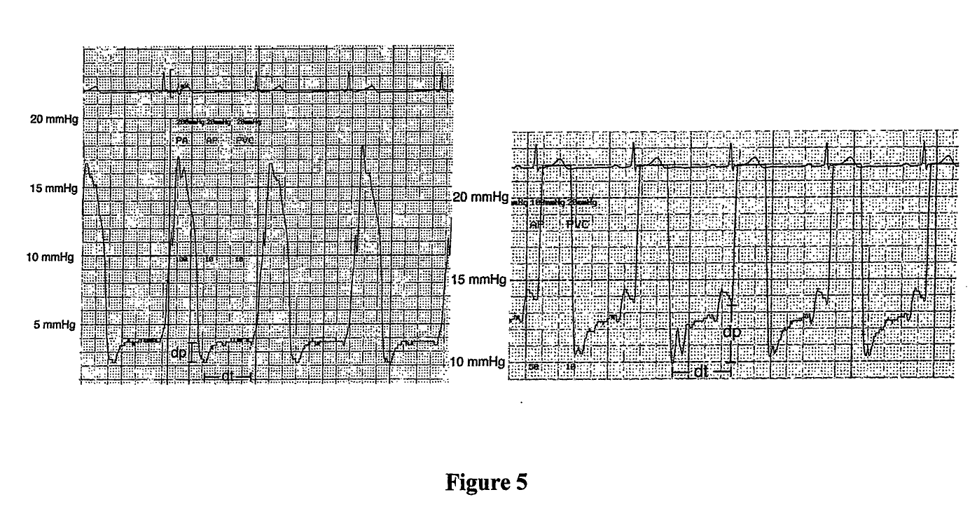

FIG. 5 shows an example of a right intraventricular pressure waveform obtained from a “normal” subject (left panel). The clinical relevance of this example is described in further details hereinbelow.

The presence of an electrocardiogram (upper curve) with the right intraventricular pressure waveform (lower curve) helps in producing the following interpretation of the intraventricular pressure waveform. The vertical line located at the beginning of the second cardiac illustrated cycle indicates the beginning of a systole. The intraventricular pressure increases rapidly and afterward, stays relatively high for a relatively small duration and subsequently decreases also rapidly. This part of the waveform is associated with the systole wherein the right ventricle contracts to eject blood. The contraction causes the increase in pressure.

In normal subjects, there is little or no substantial increase in intraventricular pressure in the diastolic phase of the cardiac cycle. This is clearly shown in the above-referenced figure wherein the intraventricular pressure increases by about 1 mmHg shortly after the end of the systole to stabilize and stay substantially constant for the rest of the diastole. This type of waveform is not necessarily observed in subjects suffering from selected pathologies.

A study was performed to establish criteria for assessing right ventricular diastolic function from right intraventricular pressure waveform. These criteria were derived by measuring various right intraventricular pressure waveform and extracting various parameters therefrom in 32 normal and 32 pathologic subjects. A trained clinician assessed normality using echocardiography. Specifically, normal subjects showed no or only mild right ventricular diastolic dysfunction while pathologic subjects showed moderate or severe right ventricular diastolic dysfunction

Two parameters that were particularly useful were an increase in right intraventricular pressure during a diastole and a slope of this increase. It was found that normal subjects had an increase in right intraventricular pressure of 3.1+/−0.8 mmHg during the diastole while pathologic subjects showed an increase in right intraventricular pressure of 5.8+/−2 mmHg during the diastole. These two groups were very significantly distinct (p<0.0001). Similarly, normal and pathologic subjects had respectively an average slope of the intraventricular pressure waveform during the diastole of 6.3+/−2.6 mmHg and 12.5+/−5.8 mmHg (p<0.0001).

Method—Description

Therefore, a method for diagnosing a right ventricular dysfunction of a subject including the following steps is suggested. First, a right intraventricular pressure waveform is measured in the subject over at least one cardiac cycle. Then, a ventricular parameter indicative of a right ventricular function is extracted from the measured waveform. Afterwards, a diagnosis is established at least in part on a basis of the ventricular parameter.

For the purpose of this document, a pressure waveform includes a plurality of pressure measurements as a function of time. Also, a parameter is any number or set of numbers obtained from a relevant data set. A pressure waveform is an example of such a relevant data set.

For example, the ventricular parameter is an increase in right intraventricular pressure during a diastole of the right ventricle. In this case, a right ventricular diastolic dysfunction is indicated by an increase of at least a first predetermined amount, for example about 4 mmHg, in right intraventricular pressure during the diastole. A more severe criterion for establishing the same diagnosis is an increase of at least about 5 mmHg in right intraventricular pressure during the diastole.

In another example, the ventricular parameter is a slope of an increase in right intraventricular pressure during a diastole of the right ventricle. In this case, a right ventricular diastolic dysfunction is indicated by a slope of right intraventricular pressure increase of at least a predetermined amount, for example about 10 mmHg/s, during the diastole. A more severe criterion for establishing the same diagnosis is an increase of at least about 11 mmHg in right intraventricular pressure during the diastole.

If criteria including intervals of right intraventricular pressure increases, or of the slopes thereof, during the diastole are used, there is a possibility of assessing a severity of a right ventricular diastolic dysfunction and to classify the right ventricular diastolic dysfunction according to a severity scale.

Examples detailed hereinbelow illustrate the above-described method.

In a variant, a pulmonary artery pressure waveform is measured in the subject over at least one cardiac cycle in addition to the right intraventricular pressure waveform. Subsequently, a pulmonary artery parameter indicative of a pulmonary artery function is extracted from the measured waveform. Then, a diagnosis is established at least in part on a basis of the pulmonary artery pressure waveform parameter and at least in part on a basis of the right intraventricular waveform parameter.

In a specific example, the right ventricular parameter is a maximal right intraventricular systolic pressure and the pulmonary artery parameter is a maximal pulmonary artery systolic pressure. Then, a diagnosis of pulmonary artery obstruction is established when the maximal pulmonary artery systolic pressure is substantially smaller than the maximal right ventricular systolic pressure. In some embodiments of the invention, a diagnosis of pulmonary artery obstruction is established when the maximal pulmonary artery systolic pressure is smaller by at least a first predetermined value, for example about 5 mmHg, than the maximal pulmonary artery systolic pressure.

In other embodiments of the invention, a diagnosis of moderate pulmonary artery obstruction is established when the maximal pulmonary artery systolic pressure is smaller by at least a second predetermined value and smaller by at most a third predetermined value than the maximal pulmonary artery systolic pressure, and a diagnosis of severe pulmonary artery obstruction is established when the maximal pulmonary artery systolic pressure is smaller by at least the third predetermined value than the maximal pulmonary artery systolic pressure. In a specific example of implementation, the second and third predetermined values are respectively about 5 mmHg and about 10 mmHg. However, other values are within the scope of the invention.

In another variant, there is provided a method for monitoring a right ventricular function of a subject. In the method, a pressure monitoring device is inserted in the right ventricle of the subject. Then, a right intraventricular pressure waveform is measured in the subject over a plurality of cardiac cycles and a ventricular parameter indicative of a right ventricular function is extracted from the measured waveform for at least some cardiac cycles from the plurality of cardiac cycles.

In another variant, the above-described pressure monitoring device is used to classify a subject as being likely to experience complications during a surgery. To that effect a right intraventricular pressure waveform is measured in the subject over at least one cardiac cycle prior to the surgery. Then, a ventricular parameter indicative of a right ventricular function is extracted from the measured waveform. Subsequently, a likelihood of occurrence of complications during the surgery is established at least in part on a basis of the ventricular parameter.

For example, the ventricular parameter is an increase in right intraventricular pressure during a diastole of the right ventricle. In this case, the likelihood of occurrence of complications during the surgery is established as being high upon the measurement of an increase of at least a first predetermined value, for example about 5 mmHg, in right intraventricular pressure during the diastole.

### Examples 1

FIG. 5, shows the effect of administering 500 ml of a colloid (Pentaspan) in two patients presenting different right intraventricular pressure (RVP) waveforms. The first patient (left-hand side of FIG. 5), who responded to the administration of the administration of the colloid by increasing an ejection volume, presents a normal right intraventricular pressure waveform with a substantially constant measured pressure during the diastole. The second patient, who did not respond to the administration, presents an increasing right intraventricular pressure waveform that increases during the diastole.

### Example 2

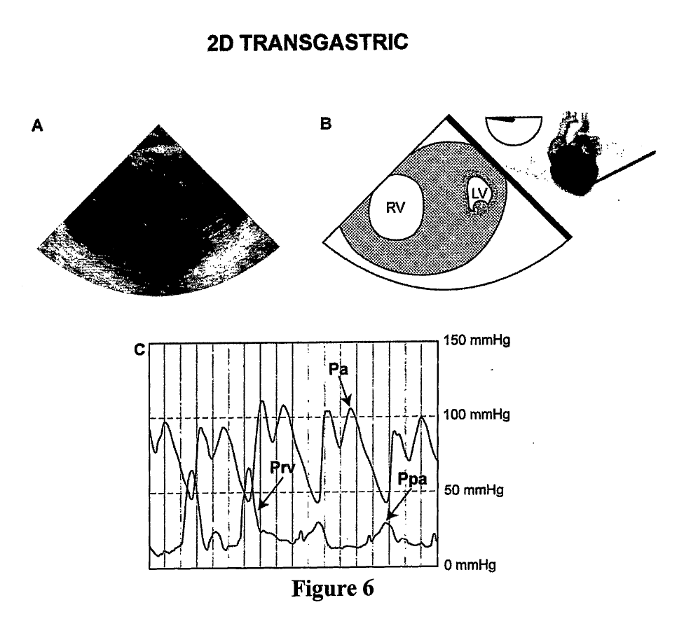

FIG. 6 illustrates measurements taken in a 75 years-old man suffering from right ventricular outflow tract obstruction after coronary revascularization and aortic valve replacement. The procedure was complicated by difficult weaning from cardiopulmonary bypass requiring intra-aortic balloon counterpulsation after a second failed attempt of weaning from the cardiopulmonary bypass. Panels A and B illustrate a trans-gastric mid-papillary short-axis echographic view (respectively with an echographic image and a segmented model obtained from the echographic image) revealing a dilated and hypertrophied right ventricle (RV). Unexplained acute right heart failure was present without pulmonary hypertension. As shown in panel C, the pulmonary artery pressure (Ppa) was 34/22 mmHg and right atrial pressure 20 mmHg. However a significant systolic pressure gradient between the right intraventricular pressure (Prv) and the pulmonary artery was present. (LV: left ventricle, Pa; arterial pressure)

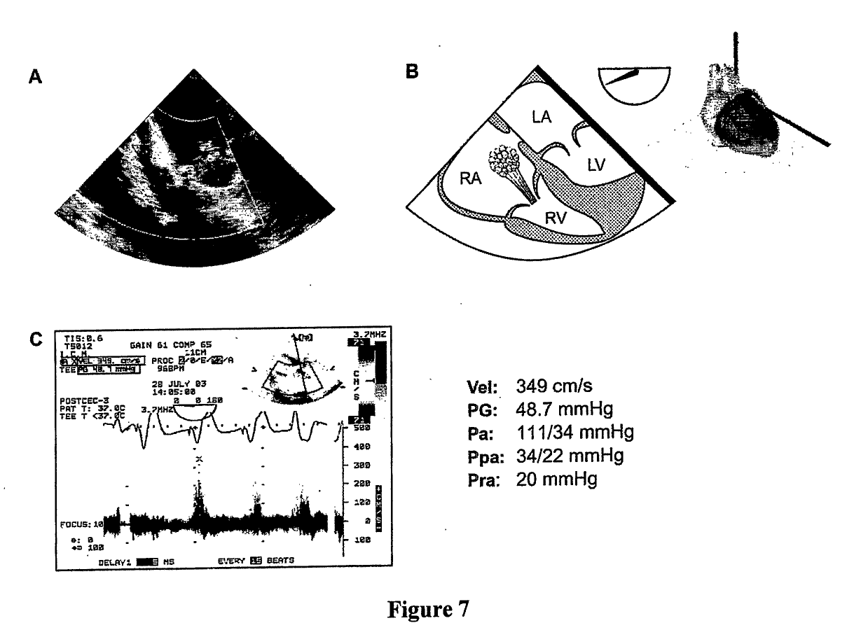

As shown in FIG. 7, for the same patient, the right ventricular systolic pressure is estimated at 68.7 mmHg based on a right atrial pressure (Pra) of 20 mmHg and a right ventricle (RV) to right atrium (RA) pressure gradient (PG) of 48.7 mmHg from a tricuspid regurgitant velocity (Vel) of 349 cm/s (panels A and B show respectively an echographic image and a segmented model obtained from the echographic image).

The pulmonary artery pressure (Ppa) was directly measured at 34/22 mmHg (systolic/diastolic). This would yield an outflow tract dynamic obstruction pressure gradient of 34.7 mmHg confirmed by directed right intraventricular pressure tracing (see FIG. 6). Panel C illustrates a continuous Doppler signal used to obtain, among other information, a pressure gradient (PG).

During surgery, the obstruction was exacerbated by intravenous milrinone and dopamine which were promptly discontinued. Weaning from cardiopulmonary bypass was then successful. The next day, all vasoactive medications were stopped and no residual right ventricular to pulmonary artery gradient was present (LA: left atrium, LV: left ventricle, Pa: arterial pressure.

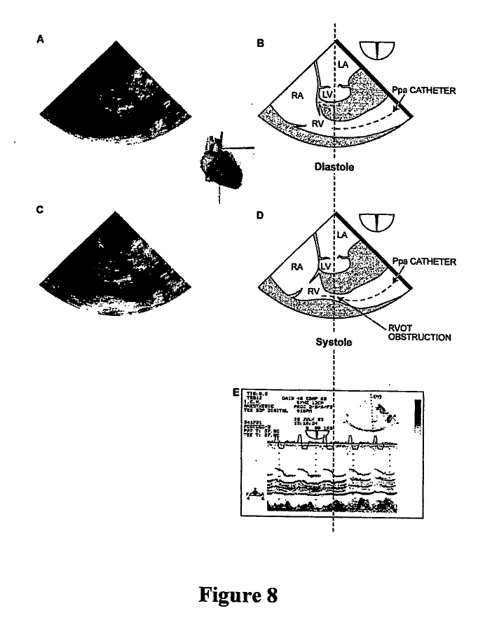

As shown in FIG. 8, a mid-oesophageal right ventricular inflow-outflow view exam showed dynamic right ventricular outflow tract (RVOT) obstruction using 2D (panels A-D representing echographic images and corresponding segmented models at the diastole (panels A and B) and at the systole (panels C and D)) and M-mode echocardiography (panel E) (LA: left atrium, LV: left ventricle, Ppa: pulmonary artery pressure, RA: right atrium, RV: right ventricle,)

### Example 3

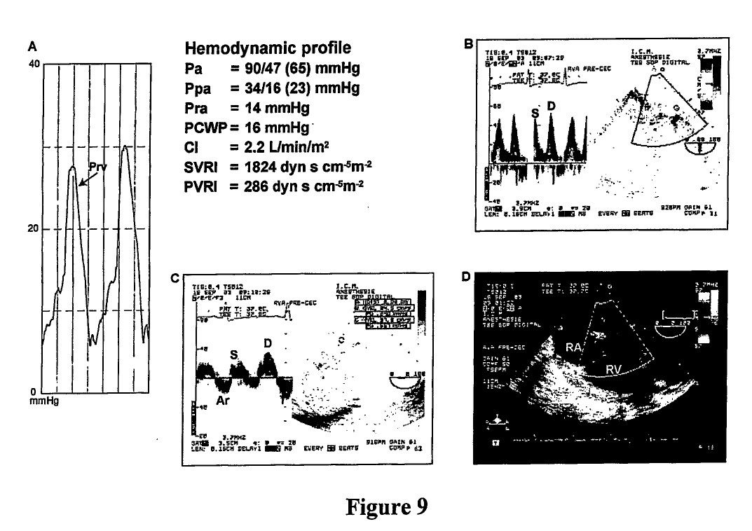

FIG. 9 shows a hemodynamic and transesophageal echocardiographic evaluation of a 46 years-old woman scheduled for aortic valve endocarditis. Despite a pulmonary artery pressure (Ppa) of 34/16 mmHg and pulmonary vascular resistance index (PVRI) at 286 dyn.s.cm-5 m-2, this patient had abnormal right intraventricular pressure (Pvr) diastolic filling waveform characterized by a rapid upstroke (Panel A illustrating the right intraventricular pressure waveform) and abnormal S/D ratio<1 in the pulmonary (panel B) and hepatic (panel C) venous flow obtained from Doppler imaging consistent with both left and right ventricular diastolic dysfunction. In addition a dilated right atrium (RA) and right ventricle (RV) were present without significant tricuspid regurgitation in a mid-oesophageal right ventricular view (panel D, which is a an echocardiographic image).

The mean arterial (MAP) to mean pulmonary artery pressure (MPAP) ratio was 65/23 or 2.8. Weaning from cardiopulmonary bypass was difficult and required noradrenaline at 200 μg/min. (Pa: arterial pressure, Pra: right atrial pressure, PCWP: pulmonary capillary wedge pressure, CI: cardiac index, SVRI: systemic vascular resistance index).

### Example 4

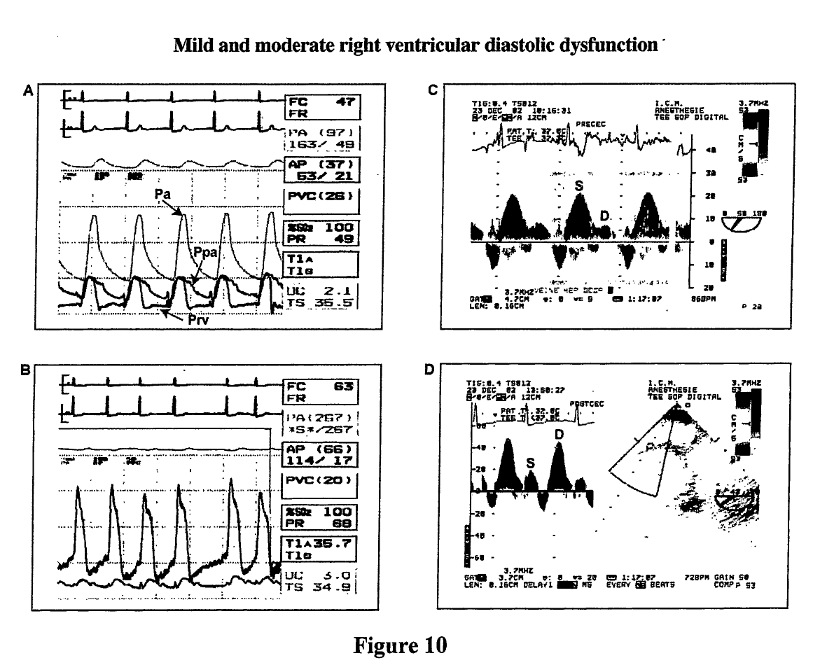

FIG. 10 illustrates the right intraventricular pressure curve and hepatic Doppler signal in an 81 years-old female scheduled for coronary revascularization and aortic and mitral valve replacement. The initial right diastolic pressure curve (pre-operative) is flat and shown with the pulmonary artery pressure waveform (panel A). After bypass, the slope of the right ventricular diastolic pressure waveform is increased (panel B). This is associated initially with a normal hepatic venous Doppler signal (panel C) that changes after cardiopulmonary bypass with predominant D to S ratio (panel D).

Separation from bypass required 7.5 μg/min of noradrenaline and vasoactive support was required for 24 hours. She survived and left the hospital after 14 days. This was compatible with a change from normal or mild right ventricular diastolic dysfunction to a moderate diastolic dysfunction.

Although the present invention has been described hereinabove by way of preferred embodiments thereof, it can be modified without departing from the spirit, scope and nature of the subject invention, as defined in the appended claims.

### LIST OF REFERENCES

1. Costachescu, T., Denault, A. Y., Guimond, J. G., et al. 2002, The hemodynamically unstable patient in the intensive care unit: Hemodynamic vs. transesophageal echocardiographic monitoring, Crit. Care Med. 30, 1214-1223.

2. Gage J, Rutman H, Lucido D, LeJemtel T H: Additive effects of dobutamine and amrinone on myocardial contractility and ventricular performance in patients with severe heart failure. Circulation 1986; 74: 367-73

3. Monrad E S, McKay R G, Baim D S, Colucci W S, Fifer M A, Heller G V, Royal H D, Grossman W: Improvement in indexes of diastolic performance in patients with congestive heart failure treated with milrinone. Circulation 1984; 70: 1030-7

4. Lobato E B, Florete O J, Bingham H L: A single dose of milrinone facilitates separation from cardiopulmonary bypass in patients with pre-existing left ventricular dysfunction. British Journal of Anaesthesia 1998; 81: 782-4

5. Lobato E B, Gravenstein N, Martin T D: Milrinone, not epinephrine, improves left ventricular compliance after cardiopulmonary bypass. J. Cardiothorac. Vasc. Anesth. 2000; 14: 374-7

6. Hardy J F, Searle N, Roy M, Perrault J: Amrinone, in combination with norepinephrine, is an effective first-line drug for difficult separation from cardiopulmonary bypass. Canadian Journal of Anaesthesia—Journal Canadien d'Anesthesie 1993; 40(6): 495-501

7. Sherry K M, Locke T J: Use of milrinone in cardiac surgical patients. Cardiovasc Drugs Ther 1993; 7: 671-5

8. Demers, P., Elkouri, S., Martineau, R., et al. 2000, Outcome with high blood lactate levels during cardiopulmonary bypass in adult cardiac operation, Ann. Thorac. Surg. 70, 2082-2086.

9. Bernard, F., Denault, A., Babin, D., et al. 2001, Diastolic dysfunction is predictive of difficult weaning from cardiopulmonary bypass, Anesth. Analg. 92, 291-298.

10. Salem, R., Denault, A. Y., Couture, P., et al. 2003, Diastolic dysfunction as an independent predictor of mortality in cardiac surgery, Can. J. Anaesth. (abstract presented at the 2003 Canadian Society meeting) (http://www.cja-jca.org/cgi/content/full/50/suppl_{—}1/A95).

11. Fortier S, DeMaria R G, Lamarche Y, Malo O, Denault A, Desjardins F, Carrier M, Perrault L P, Inhaled prostacyclin reduces cardiopulmonary bypass-induced pulmonary endothelial dysfunction via increased cyclic adenosine monophosphate levels. J Thorac Cardiovasc Surg. 2004 July; 128(1):109-16.

12. Hache, M., Denault, A., Belisle, S., et al. 2003, Inhaled epoprostenol (prostacyclin) and pulmonary hypertension before cardiac surgery, J. Thorac. Cardiovasc. Surg. 125, 642-649

13. Hache, M., Denault, A. Y., Belisle, S., et al. 2001, Inhaled prostacyclin (PGI(2)) is an effective addition to the treatment of pulmonary hypertension and hypoxia in the operating room and intensive care unit: [L'inhalation de prostacycline (PGI(2)) est un traitement complémentaire efficace de l'hypertension pulmonaire et de l'hypoxie observées en salle d'opération et à l'unité des soins intensifs], Can. J. Anaesth. 48, 924-929.

14. Robitaille, A., Denault, A., Couture, P., et al. 2003, The mean arterial to pulmonary artery pressure ratio in cardiac surgical patients is a predictor of adverse hemodynamic outcome in the intensive care unit, Can. J. Anaesth. (abstract presented at the 2003 Canadian Society meeting) (http://www.cja-jca.org/cgi/content/full/50/suppl_{—}1/A69).

15. Costachescu, T., Denault, A. Y., Guimond, J. G., et al. 2002, The hemodynamically unstable patient in the intensive care unit: Hemodynamic vs. transesophageal echocardiographic monitoring, Crit. Care Med. 30, 1214-1223.

16. Malouf, J. F., Enriquez-Sarano, M., Pellikka, P. A., et al. 2002, Severe pulmonary hypertension in patients with severe aortic valve stenosis: clinical profile and prognostic implications, J. Am. Coll. Cardiol. 40, 789-795.

17. Higgins, T. L., Estafanous, F. G., Loop, F. D., et al. 1992, Stratification of morbidity and mortality outcome by preoperative risk factors in coronary artery bypass patients. A clinical severity score, JAMA 267, 2344-2348.

18. Bossone, E., Paciocco, G., Iarussi, D., et al. 2002, The prognostic role of the ECG in primary pulmonary hypertension, Chest 121, 513-518.

19. Marcus, J. T., Vonk, N. A., Roeleveld, R. J., et al. 2001, Impaired left ventricular filling due to right ventricular pressure overload in primary pulmonary hypertension: noninvasive monitoring using MRI, Chest 119, 1761-1765.

20. Garcia M J, Thomas J D, Klein A L: New Doppler echocardiographic applications for the study of diastolic function. J. Am. Coll. Cardiol. 1998; 32: 865-75

21. Denault A, Couture P, Carrier M, Fortier A, Babin D and Tardif J C, 2004, Pre-operative moderate to severe left and right ventricular diastolic dysfunction are predictive of difficult separation from bypass. Canadian Journal of Anesthesia 51:A78 (2004).

1. A pressure monitoring device for monitoring a right intraventricular pressure in a heart having a right ventricle, the right ventricle having electrically excitable tissues and electrically non-excitable tissues, the right ventricle being in fluid communication with a pulmonary artery, said device comprising:
a. a pressure measuring portion for measuring the right intraventricular pressure, said pressure measuring portion being insertable within the right ventricle; and
b. a stabilizer connected to said pressure measuring portion for stabilising said pressure monitoring device such that when said pressure measuring portion is positioned within the right ventricle for measuring the intraventricular pressure therein, said pressure monitoring device is spaced from the electrically excitable tissues of the right ventricle.

2. A pressure monitoring device as defined in claim 1, wherein said stabilizer includes a substantially elongated and deformable stabilising body defining a stabilising body proximal end and a longitudinally opposed stabilising body distal end.

3. A pressure monitoring device as defined in claim 2, wherein said stabilising body is configured and sized such as to be located at least in part within a pulmonary artery when said pressure measuring portion is positioned for measuring the intraventricular pressure.

4. A pressure monitoring device as defined in claim 3, wherein said stabilising body distal end is configured and sized such as to be located within a pulmonary artery when said pressure measuring portion is positioned for measuring the intraventricular pressure.

5. A pressure monitoring device as defined in claim 4, wherein said stabilizer includes:
a. an inflatable balloon connected to said stabilising body and located in proximity to said stabilising body distal end; and
b. an inflation system fluidly coupled to said balloon for controllably inflating and deflating said balloon.

6. A pressure monitoring device as defined in claim 2, wherein said pressure measuring portion includes:
a. a substantially elongated and deformable pressure measurement body defining a pressure measurement body proximal end and a longitudinally opposed pressure measurement body distal end, said pressure measurement body distal end being connected to said proximal stabilising body end, said pressure measurement body having a substantially longitudinally extending lumen, said pressure measurement body also having a lateral opening in fluid communication with said lumen and extending substantially radially therefrom in a substantially proximal relationship to said pressure measurement body distal end; and
b. a pressure sensor for sensing a pressure of a fluid within said the right ventricle.

7. A pressure monitoring device as defined in claim 6, wherein said pressure sensor is located in a substantially proximal relationship to said pressure measurement body proximal end so as to be located outside of the subject when said pressure measurement portion is positioned for measuring the intraventricular pressure.

8. A pressure monitoring device as defined in claim 7, wherein said opening is substantially rectangular.

9. A pressure monitoring device as defined in claim 8, wherein said opening is oriented substantially longitudinally in said pressure measurement body.

10. A pressure monitoring device as defined in claim 9, wherein said lateral opening is located at about 25-40 cm from said stabilising body distal end.

11. A pressure monitoring device as defined in claim 10, wherein said lateral opening is located at about 30 cm from said stabilising body distal end.

12. A method for diagnosing a right ventricular dysfunction of a subject, said method comprising the steps of:
a. measuring a right intraventricular pressure waveform in the subject over at least one cardiac cycle;
b. extracting a ventricular parameter indicative of a right ventricular function from the measured right intraventricular pressure waveform; and
c. establishing a diagnosis at least in part on a basis of the ventricular parameter.

13. A method as defined in claim 12, wherein the ventricular parameter is an increase in right intraventricular pressure during a diastole of the right ventricle.

14. A method as defined in claim 13, wherein the diagnosis is a right ventricular diastolic dysfunction indicated by an increase in right intraventricular pressure during the diastole of at least a first predetermined amount.

15. A method as defined in claim 14, wherein the first predetermined amount is about 4 mmHg.

16. A method as defined in claim 14, wherein the first predetermined amount is about 5 mmHg.

17. A method as defined in claim 12, wherein the ventricular parameter is a slope of an increase in right intraventricular pressure during a diastole of the right ventricle.

18. A method as defined in claim 17, wherein the diagnosis is a right ventricular diastolic dysfunction indicated by a slope of an increase in right intraventricular pressure during the diastole larger than a first predetermined amount.

19. A method as defined in claim 18, wherein the first predetermined amount is about 10 mmHg/s.

20. A method as defined in claim 12, further comprising the steps of:
a. measuring a pulmonary artery pressure waveform in the subject over at least one cardiac cycle;
b. extracting a pulmonary artery parameter indicative of a pulmonary artery function from the measured pulmonary artery pressure waveform; and
c. establishing a diagnosis at least in part on a basis of the pulmonary artery parameter and at least in part on a basis of the intraventricular parameter.

21. A method as defined in claim 20, wherein:
a. the ventricular parameter is a maximal right intraventricular systolic pressure; and
b. the pulmonary artery parameter is a maximal pulmonary artery systolic pressure.

22. A method as defined in claim 21, wherein a diagnosis of pulmonary artery obstruction is established when the maximal pulmonary artery systolic pressure is substantially smaller than the maximal right intraventricular systolic pressure.

23. A method as defined in claim 22, wherein a diagnosis of pulmonary artery obstruction is established when the maximal pulmonary artery systolic pressure is smaller than the maximal right intraventricular systolic pressure by at least a first predetermined amount.

24. A method as defined in claim 23, wherein the first predetermined amount is about 5 mmHg.

25. A method as defined in claim 22, wherein a diagnosis of mild pulmonary artery obstruction is established when the maximal pulmonary artery systolic pressure is smaller than the maximal right intraventricular systolic pressure by at least a second predetermined amount and at most a third predetermined amount.

26. A method as defined in claim 25, wherein a diagnosis of severe pulmonary artery obstruction is established when the maximal pulmonary artery systolic pressure is smaller than the maximal right intraventricular systolic pressure by at least the third predetermined amount.

27. A method as defined in claims 25, wherein the second predetermined amount is about 5 mmHg.

28. A method as defined in claims 25, wherein the third predetermined amount is about 10 mmHg.

29. A method for monitoring a right ventricular function of a subject having a right ventricle, said method comprising the steps of:
a. inserting a pressure monitoring device in the right ventricle of the subject;
b. measuring a right intraventricular pressure waveform in the subject over a plurality of cardiac cycles; and
c. extracting a ventricular parameter indicative of a right ventricular function from the measured waveform for at least some cardiac cycles from the plurality of cardiac cycles.

30. A method as defined in claim 20, wherein:
a. the right ventricle has electrically excitable tissues and electrically non-excitable tissues;
b. the right intraventricular pressure waveform is measured with a pressure monitoring device and the pressure monitoring device includes a pressure measuring portion for measuring the right intraventricular pressure, the measuring portion being insertable within the right ventricle, and a stabilizer connected to the pressure measuring portion for stabilising the pressure monitoring device such that when the pressure measuring portion is positioned for measuring the intraventricular pressure, the pressure monitoring device is spaced from the electrically excitable tissues of the right ventricle.

31. A method for classifying a subject as being likely to experience complications during a surgery, said method comprising the steps of:
a. measuring a right intraventricular pressure waveform in the subject over at least one cardiac cycle prior to the surgery;
b. extracting a ventricular parameter indicative of a right ventricular function from the measured waveform; and
c. establishing a likelihood of occurrence of complications during the surgery at least in part on a basis of the ventricular parameter.

32. A method as defined in claim 31, wherein the ventricular parameter is an increase in right intraventricular pressure during a diastole of the right ventricle.

33. A method as defined in claim 32, wherein the likelihood of occurrence of complications during the surgery is established as being high upon the measurement of an increase in right intraventricular pressure during the diastole of at least a first predetermined amount.

34. A method as defined in claim 33, wherein the first predetermined amount is about 5 mmHg.

35. A method as defined in claims 26, wherein the second predetermined amount is about 5 mmHg.

36. A method as defined in claims 26, wherein the third predetermined amount is about 10 mmHg.
